(19) United States
(12) Patent Application Publication
(10) Pub. No.: US 2005/0234308 A1
(43) Pub. Date: Oct. 20, 2005

(54) Terminal and associated method and computer program product for monitoring at least one condition of a user

(75) Inventor: Santtu Naukkarinen, Espoo (FI)

Correspondence Address: ALSTON & BIRD LLP;BANK OF AMERICA PLAZA, 101 SOUTH TRYON STREET, SUITE 4000, CHARLOTTE, NC, 28280-4000, US

(73) Assignee: Nokia Corporation, Espoo (FI)

(21) Appl. No.: 10/827,027

(22) Filed: Apr. 19, 2004

(51) Int. Cl. A61B 5/00; A61B 5/05

(52) U.S. Cl. 600/300; 600/547

(57) ABSTRACT

A terminal is provided that includes data transfer circuitry, tactile circuitry and at least one conductor. The data transfer circuitry is capable of transferring and/or receiving data. The tactile circuitry, on the other hand, is capable of measuring at least one condition of a user. The terminal also includes at least one conductor coupled to both the data transfer circuitry and the tactile circuitry. In this regard, the conductor(s) are capable of communicating with the data transfer circuitry to function as an antenna, and capable of communicating with the tactile circuitry to function as a tactile interface. To control communication with the conductor(s), the terminal can further include switching logic capable of operating to permit the data transfer circuitry to communicate with the conductor(s) functioning as an antenna, or permit the tactile circuitry to communicate with the conductor(s) functioning as a tactile interface.

### FIELD OF THE INVENTION

The present invention generally relates to systems and methods for monitoring activities of a user and, more particularly, relates to systems and methods of monitoring and tracking fitness activities of a user.

### BACKGROUND OF THE INVENTION

People follow exercise programs for a variety of reasons. These reasons include maintaining general well-being, assisting a weight loss program and preparation for a particular sporting event, such as a marathon. Such programs need to be carefully formulated and managed if the desired effect is to be achieved, and the exerciser is to avoid injury. It is known, for example from U.S. Pat. No. 6,635,013, to use a computer to provide a user with an exercise program. However, this system merely provides printed static instructions. Consequently, a person who requires more interactive exercise program development must employ a personal fitness trainer, which can be inconvenient and costly.

Systems and apparatuses have been developed to provide a fitness program that is cost-effective and convenient. One such apparatus is disclosed by Great Britain (GB) Patent Application No. 0326387.8, entitled: Apparatus and Method for Providing a User with a Personal Exercise Program, filed Nov. 12, 2003, the contents of which are hereby incorporated by reference in its entirety. As disclosed by GB 0326387.8, an exercise assistance apparatus includes a user interface, which can comprise a wireless communication receiver, and a processor, which can comprise a mobile phone. The apparatus is configured for generating an exercise program based upon physical parameters, such as physiological information (e.g., information relating to aerobic fitness) of a user, where the exercise program can include aerobic fitness and/or strength enhancing exercises. The apparatus can also be configured for controlling the user interface to provide guidance to the user during performance of a generated program.

The apparatus can be configured to generate a program that includes a plurality of exercise definitions, each including a variable exercise duration parameter. The apparatus can set the variable parameter based upon the physiological information, such as the input information relating to aerobic fitness. The apparatus can also be configured to compute an exercise duration by multiplying a base duration by an aerobic fitness value for the user. The aerobic fitness value, in turn, can be determined based upon the input physiological information, and thereafter modified, such as at predetermined times (e.g., intervals of three to eight weeks), based upon physiological information that can be input at the end of an exercise of the generated program. More particularly, for example, the aerobic fitness value can be modified by determining an expected performance, determining actual performance from the physiological information received after exercises, comparing the expected and actual performances, and thereafter increasing or decreasing the aerobic fitness value based upon the comparison.

The apparatus can also be configured to generate a program by selecting a mix of exercises of different intensity classes, where the ratios of the mix of intensities are determined by the aerobic fitness value. If so desired, the ratios can be further determined based upon the number of exercise sessions per week in the generated program. The apparatus can be configured to select a varied selection of exercises in an intensity class from a predetermined list of exercises, such as by selecting exercises for a terminal period of the program that represent a reduction in intensity.

The apparatus can further be configured to generate a program by selecting exercises based upon a strength value, where the strength value can be determined based upon the input physiological information. In such instances, the apparatus can be configured to select exercises for the program that become successively harder during the program. And as indicated above, the apparatus can be configured to determine a varied selection of exercises from a predetermined list of exercises.

Whereas an apparatus such as that disclosed by GB 0326387.8 adequately provides a fitness program that is cost-effective and convenient, it is always desirable to improve upon such apparatuses. For example, it would be desirable to design an activity monitor capable of measuring the body fat of a user since body fat has been linked to major physical threats such as heart disease, cancer and diabetes. In this regard, various body fat measuring apparatuses have been developed to better quantify the amount of body fat as a percentage of total body mass. Although many of these apparatuses provide accurate body fat measurements, such devices tend to be expensive and offer limited functionality beyond a basic measuring capability. Providing extra functionality, however, requires consideration of the size and weight of these apparatuses since users of such apparatuses typically prefer small and lightweight devices, and as the functionality of the apparatuses increases, typically so does the number of circuits and other components required to provide the functionality. For example, providing a means for wirelessly communicating the measured body fat information, such as to an exercise assistance apparatus like that disclosed by GB 0326387.8, may require added circuitry and means for communicating the measured body fat information and/or any other information derived by the apparatus.

### SUMMARY OF THE INVENTION

In light of the foregoing background, embodiments of the present invention provide a terminal and associated method and computer program product for monitoring at least one condition, such as at least one parameter of body fat, of a user. In this regard, embodiments of the present invention are capable of monitoring the parameter(s) of body fat using tactile circuitry embodied in a portable device or structure. For example, tactile circuitry can be provided with a mobile communications device, such as a terminal, and coupled to one or more conductors, electrodes or the like capable of functioning as a tactile interface. Other physiological parameters such as, for example, parameters associated with fat mass, total body water, dehydration, body fluid, and/or body fluid balance, can additionally or alternatively be measured. Irrespective of the parameter(s), however, within the terminal, the conductors can also be coupled to data transfer circuitry. As such, not only can the conductors function as a tactile interface to the tactile circuitry, but the conductors can also function as an antenna to the data transfer circuitry. Embodiments of the present invention are therefore capable of providing added condition monitoring circuitry to a mobile terminal while reducing the size and weight of the terminal that would otherwise be imposed with the inclusion of both a tactile interface and an antenna.

According to one aspect of the present invention, a terminal is provided. The terminal includes data transfer circuitry, tactile circuitry and at least one conductor. The data transfer circuitry is capable of transferring and/or receiving data. The tactile circuitry, on the other hand, is capable of measuring at least one condition of a user. The terminal also includes at least one conductor alternately coupled to the data transfer circuitry and the tactile circuitry. In this regard, the conductor(s), which can be supported by a housing of the terminal, are capable of communicating with the data transfer circuitry to function as an antenna, and capable of alternately communicating with the tactile circuitry to function as a tactile interface. To control communication with the conductor(s), the terminal can further include switching logic coupled to the tactile circuitry, data transfer circuitry and at least one conductor. In such instances, the switching logic is capable of operating to permit the data transfer circuitry to communicate with the conductor(s) functioning as an antenna, or permit the tactile circuitry to communicate with the conductor(s) functioning as a tactile interface.

The terminal can further include a controller coupled to the switching logic and capable of controlling operation of the switching logic. The controller can be capable of controlling the switching logic to permit the tactile circuitry to communicate with the conductor(s) functioning as a tactile interface when the tactile circuitry is set to operate. The controller can be capable of determining when the tactile circuitry is set to operate based upon user input and/or a context of the terminal and/or user. In this regard, the controller can also be capable of determining the context of the terminal and/or user based upon data representative of a change in a radio frequency field proximate the conductors, and/or data representative of a change in capacitance across at least one pair of conductors.

In operation, the tactile circuitry can be capable of communicating monitoring signals to a portion of the user's body via the conductor(s) operating as a tactile interface such that condition(s) of the user are capable of being computed in response to communication of the monitoring signals, such as by the tactile circuitry or the controller. The monitoring signals can comprise any of a number of different types of signals. For example, the monitoring signals can comprise sinusoidal or square wave current or voltage signals. The monitoring signals can comprise a current or voltage signal having a frequency of at least 50 kHz. Additionally or alternatively, the monitoring signals can comprise a current signal having an amperage less than 1 mA. Irrespective of the type of monitoring signals, the tactile circuitry can be capable of maintaining the monitoring signals at a substantially constant amperage or voltage.

Also irrespective of the monitoring signals, however, the tactile circuitry can be capable of communicating monitoring signals to a portion of the user's body such that a resistance to the communication of the monitoring signals is capable of being computed. In such instances, at least one parameter associated with the user's body fat can be computed based upon the resistance. For example, a percent body fat, fat mass and/or total body water can be computed based upon the resistance.

According to other aspects of the present invention, a method and computer program product are provided for monitoring at least one condition of a user. Embodiments of the present invention therefore provide a terminal, method and computer program product for monitoring at least one condition of a user. The terminal of embodiments of the present invention includes multifunctional conductor(s) capable of function as either a tactile interface for tactile circuitry or an antenna for data transfer circuitry. Operating as a tactile interface, for example, the conductors can facilitate the tactile circuitry in communicating monitoring signals to a portion of the user's body such that condition(s) of the user are capable of being computed in response to communication of the monitoring signals. By permitting the conductor(s) to also function as an antenna, embodiments of the present invention can operate within a terminal having a reduced size and weight as compared to designs requiring both a tactile interface and an antenna. Therefore, the terminal and associated method and computer program product of embodiments of the present invention solve the problems identified by prior techniques and provide additional advantages.

### BRIEF DESCRIPTION OF THE DRAWINGS

Having thus described the invention in general terms, reference will now be made to the accompanying drawings, which are not necessarily drawn to scale, and wherein:

FIG. 1 is a schematic block diagram of a terminal of one embodiment of the present invention;

FIGS. 2A-2E are schematic illustrations of a terminal placed in proximity to a user, in accordance with various embodiments of the present invention;

FIGS. 3A and 3b are schematic front and back views of a terminal including one or more electrodes, in accordance with one embodiment of the present invention;

FIGS. 4A-4D are schematic illustrations of tactile circuitry of a terminal, in accordance with various embodiments of the present invention;

FIG. 5 is a flowchart illustrating various steps in a method of monitoring at least one condition of a user, in accordance with one embodiment of the present invention;

FIG. 6 is a schematic illustration of a terminal user grasping a terminal during operation of the terminal, in accordance with one embodiment of the present invention; and

FIG. 7 is a schematic block diagram of a wireless communications system according to one embodiment of the present invention including a mobile network and a data network to which a terminal is bi-directionally coupled through wireless RF links.

### DETAILED DESCRIPTION OF THE INVENTION

The present invention now will be described more fully hereinafter with reference to the accompanying drawings, in which preferred embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art. Like numbers refer to like elements throughout.

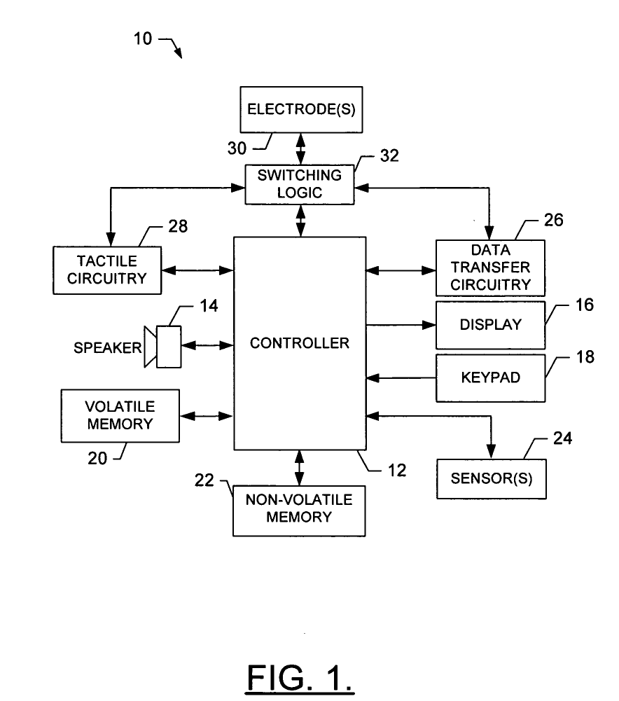

FIG. 1 illustrates a schematic block diagram of a terminal 10 in accordance with one embodiment of the present invention. It should be understood, that the terminal illustrated and hereinafter described is merely illustrative of one type of terminal that would benefit from the present invention and, therefore, should not be taken to limit the scope of the present invention. While several embodiments of the terminal are illustrated and will be hereinafter described for purposes of example, other types of terminals, such as mobile telephones, portable digital assistants (PDAs), pagers, and other types of voice and text communications systems, can readily employ the present invention.

As shown, the terminal 10 includes a processor such as a controller 12. The controller includes the circuitry required for implementing the functions of the terminal in accordance with embodiments of the present invention, as explained in greater detail below. For example, the controller may be comprised of a digital signal processor device, a microprocessor device, and/or various analog to digital converters, digital to analog converters, and other support circuits. The control and signal processing functions of the terminal are allocated between these devices according to their respective capabilities. The controller may also include the functionally to operate one or more software applications. In addition to the controller, the terminal also includes a user interface that may include, for example, a conventional earphone or speaker 14 capable of being driven by the controller to present various audible tones during operation of the terminal. The user interface may also include a display 16 and a user input interface, both of which are also coupled to the controller. The user input interface, which allows the terminal to receive data, can comprise any of a number of devices allowing the terminal to receive data, such as a keypad 18, a touch display (not shown) or other input device. In embodiments including a keypad, the keypad can include one or more keys used for operating the terminal.

The terminal 10 can also include memory, such as a volatile memory 20 and/or non-volatile memory 22. The non-volatile memory, for example, can comprise embedded or removable multimedia memory cards (MMC's), Memory Sticks manufactured by Sony Corporation, EEPROM, flash memory, hard disk or the like. The memories can store any of a number of pieces of information and data used by the terminal to implement the functions of the terminal. For example, as explained further below, the memories can store activity detection application 23 capable of operating on the terminal to monitor the fitness activities of a user of the terminal, and manage the user's personal fitness goals. In this regard, the memories can also store a database 25 including, for example, personal information regarding a user of the terminal, such as date of birth, gender, height and/or weight, as well as a step length for the user when walking and/or running. In addition, for example, the database can include personal fitness goals of the user, such as a one-time and/or weekly goal for an amount of time performing one or more activities, a number of steps take in performing the activit(ies), a number of calories burned in performing the activit(ies), and/or a distance traveled in performing the activit(ies). Likewise, for example, the database can include an amount of time spent by the user in performing one or more activities for a given time period, a number of steps taken in performing the activit(ies), a number of calories burned in performing the activit(ies), and/or a distance traveled in performing the activit(ies).

The terminal can also include one or more sensors 24 for sensing the ambient conditions of the terminal, where the conditions may be representative of the ambient conditions of the user of the terminal. In this regard, the terminal may include sensors such as, for example, a positioning sensor, a touch sensor, an audio sensor, a compass sensor, an ambient light sensor, and/or an ambient temperature sensor. The positioning sensor can comprise, for example, a global positioning system (GPS) sensor. Additionally, or alternatively, the positioning sensor can comprise, for example, a radio beacon triangulation sensor that determines the location of the wireless device by means of a network of radio beacons, base stations, or access points, as is described for example, in Nokia European patent EP 0 767 594 A3, entitled: Terminal Positioning System, published on May 12, 1999, the contents of which are hereby incorporated by reference in its entirety. Although the terminal can include any of a number of different sensors, in one typical embodiment, at least one of the sensors comprises a two or three-axis acceleration sensor (accelerometer).

In addition, the terminal can include data transfer circuitry 26, which can be coupled to the controller 12, and be capable of transferring data to and/or receiving data from electronic devices in accordance with one or more of any of a number of different wireline and/or wireless techniques. For example, the data transfer circuitry can include a radio frequency (RF) transceiver such that the terminal can share and/or obtain data in accordance with radio frequency techniques. In addition or in the alternative, the data transfer circuitry can include a Bluetooth (BT) transceiver such that the terminal can share and/or obtain data in accordance with Bluetooth transfer techniques. Further, for example, the data transfer circuitry can include transmitters, receivers and/or transceivers for transmitting and/or receiving in accordance with one or more of a number of different wireline and/or wireless networking techniques, including LAN and/or WLAN techniques.

The terminal 10 can further include tactile circuitry 28, which can be coupled to the controller 12, and be capable of monitoring one or more conditions of the terminal user, such as the body fat of the terminal user or whether the terminal user is merely contacting the terminal. In accordance with embodiments of the present invention, both the tactile circuitry and the data transfer circuitry 26 can be coupled to at least one conductor, such as at least one electrode 30, which can also be coupled to the controller. The electrode(s) are capable of functioning as a tactile interface for contacting the user's body to sense and/or measure one or more conditions of the terminal user, where data representative of the conditions can thereafter be provided to the tactile circuitry. In addition, the electrode(s) are capable of functioning as an antenna for facilitating the data transfer circuitry in transmitting data to and/or receiving data from electronic devices.

To permit the electrode(s) 30 to function as either an antenna or a tactile interface at any given time, the terminal can further include switching logic 32 coupled between the data transfer circuitry 26 and the electrode(s), and between the tactile circuitry 28 and the electrode(s). In this regard, the switching logic can operate to permit the data transfer circuitry to communicate with the electrode(s) functioning as an antenna, or to permit the tactile circuitry to communicate with the electrode(s) functioning as a tactile interface. It should be noted that while the data transfer circuitry, tactile circuitry and switching logic are shown and described herein as comprising hardware separate from the controller 12, one or more of the data transfer circuitry, tactile circuitry and switching logic can alternatively be embodied in firmware, software or the like without departing from the spirit and scope of the present invention. Also, the functions of one or more of the data transfer circuitry, tactile circuitry and switching logic can alternatively be included within the controller.

The switching logic 32 can be operated to permit the data transfer circuitry 26 or tactile circuitry 28 to communicate with the electrode(s) in any of a number of different manners. In one typical embodiment, for example, the switching logic can also be coupled to the controller 12 such that the controller can control operation of the switching logic based upon operation of the tactile circuitry. In such instances, the switching logic can be operated by default to permit the data transfer circuitry to communicate with the electrode(s). Then, during, or just prior to, operation of the tactile circuitry to monitor condition(s) of the terminal user, the controller can operate the switching logic to permit the tactile circuitry to communicate with the electrode(s).

To facilitate the controller 12 operating the switching logic 32 during operation of the tactile circuitry 28, the controller can determine when the tactile circuitry is set to operate, in accordance with any of a number of different techniques. For example, the tactile circuitry can receive terminal user input, such as from the user input interface (e.g., a keypad 18) of the terminal 10, to thereby begin monitoring the condition(s) of the terminal user. In such instances, the controller 12 can receive the user input to thereby determine that the tactile circuitry is set to operate to monitor the condition(s) of the terminal user. For example, the controller can directly receive the user input, operate the switching logic, and pass the user input to the tactile circuitry such that the tactile circuitry can thereafter operate in accordance with the user input. Alternatively, for example, the tactile circuitry can receive the user input, and communicate the user input to the controller such that the controller can determine that the tactile circuitry set to operate to monitor the condition(s) of the terminal user. In yet another alternative, for example, the controller can receive the user input from the user input interface as the tactile circuitry also receives the user input to thereby set the tactile circuitry to operate.

In addition to, or in lieu of, the controller 12 determining if the tactile circuitry 28 is set to operate based upon terminal user input from the user input interface, the controller can determine if the tactile circuitry is set to operate based upon a context of the terminal 10 and/or terminal user, the context being representative of setting the tactile circuitry to operate to monitor condition(s) of the terminal user. For example, the controller can receive, from the electrode(s) 30 and/or one or more of the sensor(s) 24, data representative of a change in the radio frequency (RF) field proximate the electrode(s) or sensor(s), or representative of a change in capacitance across one or more pairs of electrodes or sensors. Such data, in turn, can be indicative of one or more of the electrodes being in physical contact with a portion of the terminal user's anatomy in a manner such that the terminal user desires for the tactile circuitry to monitor condition(s) of the terminal user. In such instances, then, the controller can determine that the tactile circuitry is set to operate and, in turn, operate the switching logic to permit the tactile circuitry to communicate with the electrode(s).

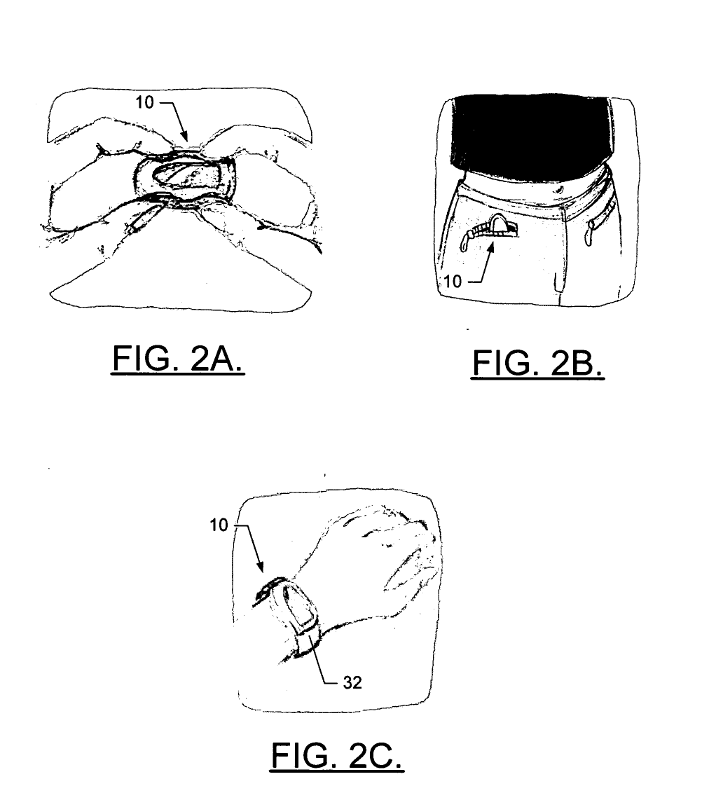

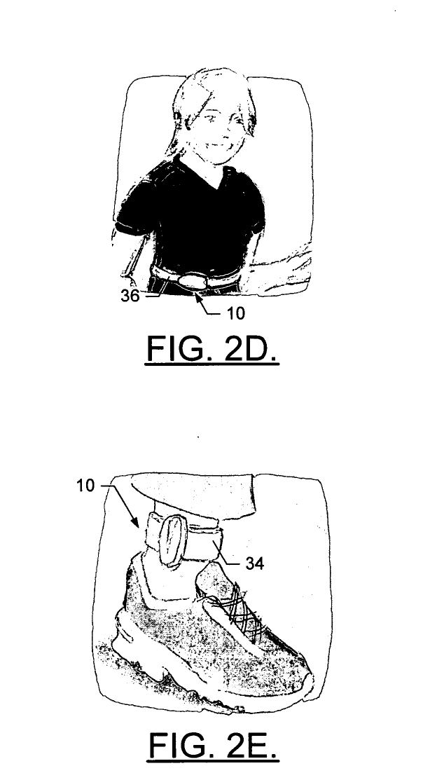

As indicated above, and shown in FIG. 2A, the terminal 10 of embodiments of the present invention is capable of being embodied in a portable package. The terminal can therefore be placed in relatively close proximity to the user. As shown in FIG. 2B, for example, the terminal can be carried in a pocket of clothing of the user. Alternatively, the terminal can be belted or otherwise strapped to a wrist, waist or ankle of the user, as shown in FIGS. 2C, 2D and 2E, respectively. In yet a number of other alternatives, for example, the terminal can be belted or otherwise strapped to an arm or leg of the user, hung from the user's neck, or clipped to clothing of the user. As will be appreciated, in many instances of placing the terminal in close proximity to the user, the terminal can include added functionality desirable for use by the user. For example, the terminal can include a clock such that the terminal can additionally or alternatively operate as a watch. As will also be appreciated, in such instances, the terminal can additionally include a strap, belt, clip, lanyard or the like. For example, as shown in FIGS. 2C and 2E, when the terminal is strapped to the wrist or ankle of the user, the terminal can be embodied in a portable package that includes a wrist strap 32 or an ankle strap 34, both of which can comprise the same strap. Also, for example, as shown in FIG. 2D, when the terminal is belted around the waist of the user, the terminal can be embodied in a portable package that includes a belt 36.

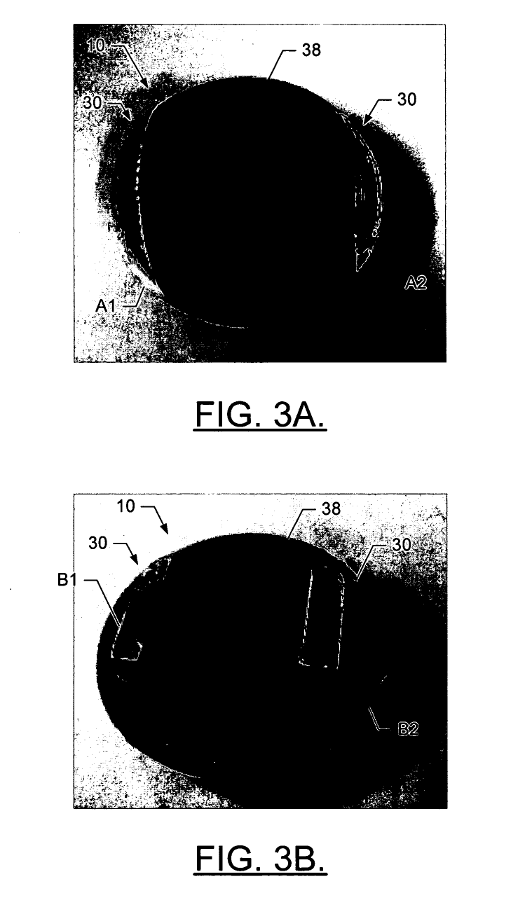

Reference is now made to FIGS. 3A and 3B, which illustrate a front and a back view of a terminal 10 of one embodiment of the present invention. As shown, the terminal includes a housing 38 that incorporates a number of electrodes 30. The electrodes can be mounted in any of a number of different manners. As shown, for example, one or more electrodes can be mounted flush with a major outer surface of the housing, and one or more electrodes can be mounted along one or more edges of the housing. Although the electrodes are shown mounted to a housing of the terminal, it should be understood that the terminal can additionally or alternatively include a cover (not shown) having one or more electrodes mounted thereon. In such instances, the cover can include electrical contacts that connect with corresponding electrical contacts on the housing when the cover is situated on the housing for use in accordance with embodiments of the present invention. In accordance with a four point probe technique for performing body fat percent measurements (described below) employing a terminal of embodiments of the present invention, the housing can include, for example, four electrodes.

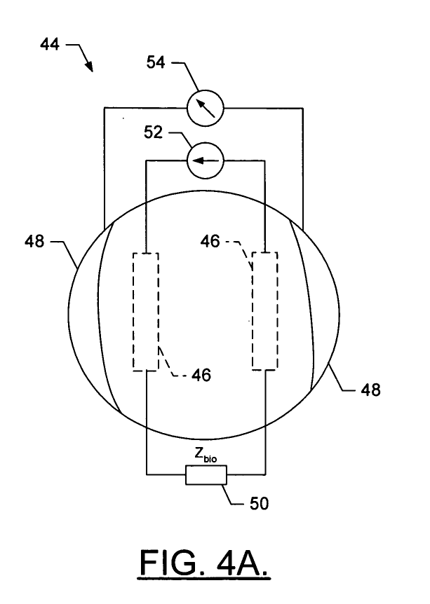

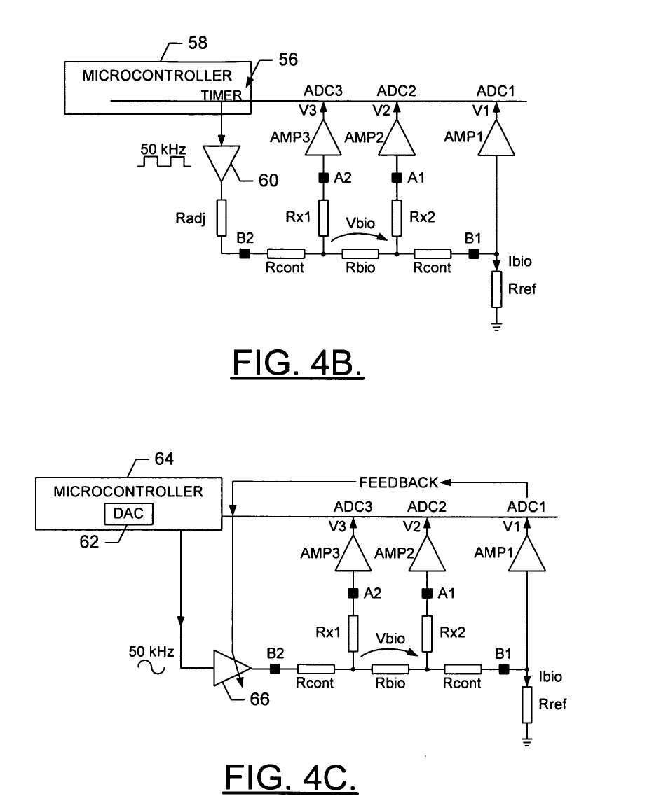

Exemplar configurations of the tactile circuitry 28 and operation of the tactile circuitry in monitoring the body fat (i.e., condition) of the terminal user will now be described in accordance with embodiments of the present invention. Referring now to FIG. 4A, there is illustrated tactile circuitry 44 of a terminal 10 implemented to perform body fat percentage measurements using a four point probe technique in accordance with an embodiment of the present invention. Although not shown, in operation, the tactile circuitry is typically in physical contact with a portion of a user's anatomy. According to a four point probe technique, two electrodes 46 serve as source electrodes and two electrodes 48 serve as detection electrodes. In general terms, body fat can be determined by measuring the resistance 50 of the body to an injected monitoring signal using a four point probe technique.

The tactile circuitry 44 includes a monitoring signal generator 52, which can comprise a current or voltage source that provides a constant current or voltage monitoring signal. The monitor signal generator can be coupled to the two source electrodes 46. It can be appreciated that a battery of the terminal 10 can provide the requisite power to the tactile circuitry. The tactile circuitry can further include a voltage detector 54 which is coupled between the two detection electrodes 48. The voltage detector typically receives an input reference signal from the monitoring signal generator.

The monitoring signal generator 52 can generate an AC drive current signal. The drive current signal can comprise, for example, a sinusoidal signal or a square wave. The frequency of the drive current signal can be, for example, approximately 50 kHz or greater. It is noted that the frequency of the drive current signal can be varied and that body fat measurements can be made at each of a number of different frequencies. The drive current signal can have an amperage, for example, less than about 1 mA, or more particularly, between approximately 0.3 mA and approximately 0.8 mA.

In normal operation, a monitoring signal generated by the monitoring signal generator 52 can be injected into the terminal user's body via the source electrodes 46. A current field can then be produced between the source electrodes in response to propagation of the monitoring signal into the user's body tissue. In this regard, the detection electrodes 48 are typically arranged such that the current field is detectable. As such, a sense voltage can be developed between the detection electrodes and measured by the voltage detector 54.

An impedance, Z_{bio}, can then be derived using the sense voltage and source current amperage. In this regard, the derived impedance value can be reflective of a biological resistance and reactance (i.e., bioimpedance, Z_{bio}) measurable between the detection electrodes 48 in response to the monitoring signals which were injected into biological tissue by the source electrodes 46. The resistance accounts for more than 95% of the biological impedance value. As such, the biological resistance component of the bioimpedance can be used to derive body fat parameters, which results in a simplified design with very good accuracy. In general, a larger detected resistance 50 is indicative of a greater amount of body fat.

FIG. 4B is a schematic of tactile circuitry which employs square wave monitoring signals in accordance with an embodiment of the present invention. In FIG. 4B, elements A1, A2, B1 and B2 represent electrodes (B1 and B2 are source electrodes, and A1 and A2 are detection electrodes in this configuration), R_{cont} represents contact resistance, Rₓ₁ and Rₓ₂ represent bioresistances in the hand, and R_{bio} represents the bioresistance that is measured and indicative of body fat. According to this embodiment, a timer 56, which is under control of a microcontroller 58, can generate a square wave drive signal. The drive signal can then be passed to the source or drive electrode B2 via a buffer amplifier 60 and resistor R_{adj}. For example, the timer can generate a square wave drive signal (I_{bio}) having a frequency of approximately 50 kHz or greater. Resistor R_{adj} can then be used to set the amperage ranging between approximately 0.4 mA and approximately 0.8 mA. The square wave generator is typically configured as a constant current source.

Voltages V₃, V₂, and V₁ represent voltages developed at the respective outputs of instrument amplifiers AMP₃, AMP₂ and AMP₁ in response to injection of the monitoring signal into the user's hands. Voltages V₃, V₂ and V₁ can be input to respective analog-to-digital converters ADC₃, ADC₂ and ADC₁. The respective digital voltage signals corresponding to analog voltages V₃, V₂ and V₁ can then be input to the microcontroller 58. The microcontroller can then compute the bioresistance, R_{bio}. Alternatively, the controller 12, or more particularly the activity detection application 23, of the terminal 10, can compute the bioresistance, R_{bio}. Irrespective of the component computing the bioresistance, the bioresistance can be computed, for example, using the following equation:

$${R}_{bio} = \frac{{V}_{bio}}{{I}_{bio}} = {R}_{ref} \cdot \frac{{V}_{3} - {V}_{2}}{{V}_{1}} \qquad ( 1 )$$

where, V_{bio} represents the voltage drop between detection electrodes A2 and A1, R_{ref} represents the reference resistance, and I_{bio} represents the drive current passing through the body from the hands via the source electrodes B2 and B1. The value of I_{bio} can be determined by measuring the voltage across the resistor R_{ref}. It can be seen from equation (1) above that employment of a four point probe technique can eliminate the contact resistance, R_{cont}, from the body fat measurement. It should also be understood that whereas the circuit components are shown as being separate from one another, two or more of the circuit components (e.g., the timer 56, amplifier 60, instrument amplifiers AMP₁-AMP₃, and A/D converters ADC₁-ADC₃, etc.) can be alternatively integrated as part of the microcontroller 58. It should also be understood that whereas the various computations, measures, determinations or the like can be performed by the microcontroller, the controller 12, or more particularly the fitness finder application 23, can be equally capable of performing the same computations, measures, determinations or the like.

FIG. 4C is a schematic of tactile circuitry that employs sinusoidal monitoring signals in accordance with another embodiment of the present invention. According to this implementation, a sinusoidal drive signal can be generated at the output of a digital-to-analog converter (DAC) 62 under control of a microcontroller 64. The drive current signal, I_{bio}, can have a frequency of at least 50 kHz and an amperage ranging between approximately 0.4 mA and approximately 0.8 mA.

The drive current signal produced at the output of DAC 62 can then be applied to an input of an instrument amplifier 66. Feedback can also be added to insure that the drive current, I_{bio}, is kept substantially constant at a predetermined amperage, such as at approximately 0.6 mA. As shown, the circuitry of FIG. 4C operates in the manner described above with regard to the circuitry of FIG. 4B in response to the application of a sinusoidal drive current, I_{bio}. In this regard, the bioresistance, R_{bio}, can be computed using equation (1) above.

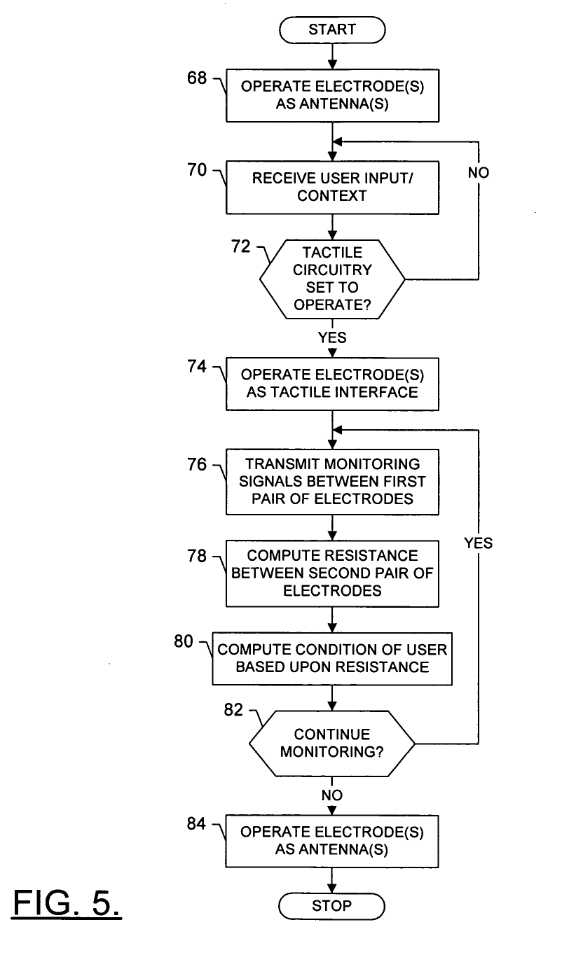

Reference is now made to FIG. 5, which illustrates various steps in a method of monitoring a condition of a terminal user in accordance with one embodiment of the present invention, the condition being one or more body fat parameters of the terminal user. As shown in block 68, the method includes operating the electrode(s) 30 as one or more antennas for communicating in accordance with one or more communication techniques. For example, the electrode(s) can be operated as antenna(s) by operating the switching logic 32 to permit the data transfer circuitry 26 to communicate with the electrode(s). In this embodiment, the electrode(s) can, by default, be operated as antenna(s). It should be understood that the electrode(s) can be operated as a tactile interface by default, or not otherwise have a default operation.

As the electrode(s) 30 are operating as antenna(s), the controller 12 can receive user input or a context of the terminal 10 or terminal user, as shown in block 70. The controller then determines that, based upon the user input/context, the tactile circuitry 28 is set to operate, as shown in block 72. If the tactile circuitry is set to operate, the electrode(s) can then be made to operate as a tactile interface, as shown in block 74. In this regard, the controller can operate the switching logic to permit the tactile circuitry to communicate with the electrode(s).

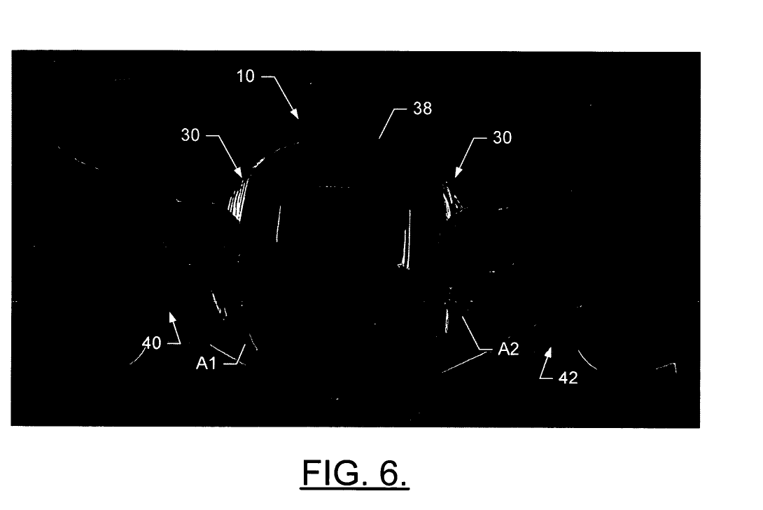

As shown in FIG. 6, for example, the terminal 10 can include two electrodes 30, A1 and A2, mounted to opposing sides of the housing 38, and two electrodes, B1 and B2, mounted on a back surface of the housing. The location of electrodes A1-A2 and B1-B2 on the terminal housing can be important for purposes of understanding the various body fat measuring techniques described herein, for example. In this regard, when the tactile circuitry is set to operate in accordance with a four point probe technique, a terminal user can grasp the terminal such that the user's index finger of the left hand 40 contacts electrode B1, and the user's index finger of the right hand 42 contacts electrode B2. The mobile terminal is grasped such that the user's left thumb contacts electrode A1, and the user's right thumb contacts electrode A2.

According to the measuring technique of FIG. 4, a monitoring signal source vector can employ electrodes B1 and B2, with a monitoring signal detection vector employing electrodes A1 and A2. In operation, then, monitoring signals can be transmitted between a first pair of electrodes 30, with a resistance thereafter detected between a second pair of electrodes. More particularly, a monitoring signal, I_{bio}, can be injected into the user's left thumb via the A1 electrode, propagate through the body, and return to the terminal 10 via the A2 electrode. The bioresistance, R_{bio}, can then be computed, such as in accordance with equation (1) above.

After computing the bioresistance, R_{bio}, a condition of the terminal user, such as body fat parameter(s) of the terminal user, can be computed, as shown in block 80. The body fat parameter(s) can be computed in any of a number of different manners, but in one embodiment, the tactile circuitry 28 computes the bioresistance, R_{bio}, from an equation that can be derived from a known formula referred to as Lukaski's and Bolonchuk's formula (see Lukaski & Bolonchuk, AVIATION, SPACE AND ENVIRONMENTAL MEDICINE, 59, pp. 1163-1169 (1988)). According to this approach, total body water (TBW), which is a measure of all of the water in a user, both intracellular and extracellular, can be computed as follows:
TBW=0.372(S²/R)+3.05(Sex)+0.142(W)−0.069(Age) (2)
where S represents stature in centimeters, R represents bioresistance in Ohms, W represents body weight in kilograms, Sex equals one for males and zero for females, and Age represents the age of the user in years.

Using total body weight, the fat free mass (FFM) of the user, in kilograms, can be computed as:

$$FFM = \frac{TBW}{0.73} \qquad ( 3 )$$

Fat free mass (FFM) yields fat mass (FM), in kilograms, can be computed as follows:
FM=Weight−FFM (4)
Percent body fat of the user can then be calculated using the following equation:

$$\% Fat = \frac{FM}{Weight} \cdot 100 \qquad ( 5 )$$

An equation derived from Lukaski's and Bolonchuk's formula, as characterized in equations (2) through (5) above, can be of particular use when performing body fat measurements with tactile circuitry implemented with a terminal 10. Because a terminal is held by users in a particular way, there is additional resistance from the users' fingers. Equation (6) below is derived from Lukaski's and Bolonchuk's formula by linearizing the 1/R term and adding a term proportional to the stature of the user squared, as follows:

$$FP = A \cdot {S}^{2} \cdot R \cdot \frac{1}{W} + B \cdot {S}^{2} \cdot \frac{1}{W} + C \cdot N \cdot \frac{1}{W} + D \cdot \frac{1}{W} + \\ E \cdot {S}^{2} + F \qquad ( 6 )$$

where FP represents fat percentage of the user, and N represents age of the user in years. It is noted that in equation (6), N can typically range between 18 and 80.

The values of parameters A through F can vary as a function of gender depending on various properties of measurement geometry. For example, the following values of parameters A through F can be used for particular measurement geometries:

TABLE 1

| Parameter | Males | Females |
|---|---|---|
| A | 2.178 × 10⁻⁴ | 1.185 × 10⁻⁴ |
| B | −0.3710 | −0.3121 |
| C | 30.80 | 25.92 |
| D | 27.26 | 0.5282 |
| E | 6.565 × 10⁻⁴ | 9.210 × 10⁻⁴ |
| F | 64.20 | 70.09 |

It is noted that equation (6) or one similar to equation (6) or Lukaski's and Bolonchuk's equation can typically be used to determine the fat percentage of persons younger than 18 years by suitable alteration of parameters A-F.

Irrespective of how the terminal user's body fat percentage is computed, thereafter the controller can determine if the tactile circuitry 28 is continuing to monitor the condition of the terminal user, such as by again determining if the tactile circuitry is set to operate, as shown in block 82. If the tactile circuitry is set to operate, the method can continue, such as by again transmitting monitoring signals between the first pair of electrodes (see block 76). If the tactile circuitry is no longer set to operate, the electrode(s) 30 can again be operated as antenna(s), as shown in block 84. For example, the controller 12 can again operate the switching logic 32 to permit the data transfer circuitry 26 to communicate with the electrode(s).

As explained above, the electrode(s) 30 can be alternately coupled to the data transfer circuitry 26 and the tactile circuitry 28. It should be understood, however, that in various instances the data transfer circuitry may be communicating with the electrode(s) when the tactile circuitry attempts to communicate with the electrode(s), or the tactile circuitry may be communicating with the electrode(s) when the data transfer circuitry desires to communicate with the electrode(s). In such instances, the terminal 10 can be configured to respond in any of a number of different manners. For example, the controller 12 of the terminal can be configured to inform the terminal user that the data transfer circuitry or tactile circuitry (i.e., second component) attempting communication with the electrode(s) has been prevented from doing so because the resource (i.e., electrode(s)) are currently in use by the other of the data transfer circuitry and tactile circuitry (i.e., first component). The controller can then continue, if so desired, by informing the terminal user that operation of the first component should be terminated to allow the second component to communicate with the electrode(s), or otherwise function for its intended purpose.

In addition to providing body fat measuring capability, the terminal 10 of embodiments of the present invention can be capable of monitoring other activities of a terminal user. As indicated above, the terminal can store an activity detection application 23 capable of operating on the terminal to monitor the fitness activities of a user of the terminal, and manage the user's personal fitness goals. In this regard, the activity detection application is capable of interfacing with the sensor(s) 24 of the terminal to receive measurement(s) of the ambient condition(s) of the user, such as to receive acceleration measurements indicative of movement over a distance for one or more periods of time. In this regard, the movement may be representative of the user taking one or more steps while performing one or more activities over those period(s) of time. As the activity detection application receives such measurement(s), the activity detection application can be capable of tracking the duration of activity of the user, the distance moved by the user in performing the activity, the number of steps taken by the user of the distance, and/or the speed of movement of the user. The activity detection application can additionally be capable of computing energy (e.g., calories) expended by the user in performing the activity. For more information on operation of such an activity detection application, see U.S. patent application Ser. No.______entitled: SYSTEM AND ASSOCIATED TERMINAL, METHOD AND COMPUTER PROGRAM PRODUCT FOR MONITORING AT LEAST ONE ACTIVITY OF A USER, filed ______, the contents of which are hereby incorporated by reference in its entirety.

As the tactile circuitry 28 computes the body fat percentage of the terminal user, the tactile circuitry, or more typically activity detection application 23, can record the body fat percentage, such as in the database of the terminal. Similarly, as the activity detection application operates and determines or computes the various values, the activity detection application can record one or more values, such as in the database of the terminal 10. For example, the activity detection application can record the energy expended, duration, distance and/or detected steps for the user in performing the selected activity.

The values recorded by the activity detection application 23 can thereafter be compared to goals of the user. For example, the recorded body fat percentage and with respect to activities of the user, energy expended, duration, distance and/or detected steps can be compared to goals for body fat percentage, energy expended, duration, distance and/or detected steps, respectively. In this regard, the terminal user can input, and the activity detection application can receive, goals of the user. For example, the activity detection application can receive goals such as a desired body fat percentage and with respect to one or more selected activities, amount of energy expended, duration of performing an activity, distance over which to perform the activity and/or number of steps in performing the activity. As will be appreciated, the goals can be received for any of a number of different time periods, such as over a day, week, month, year, etc. In addition to the values recorded over a given time period, and/or the goals for the respective values of the given time period, the activity detection application 23 can be capable of presenting the comparison of the goals of the user and the user's progress toward those goals. For more information on managing personal fitness goals of the terminal user, see U.S. patent application Ser. No.______entitled: SYSTEM AND ASSOCIATED TERMINAL, METHOD AND COMPUTER PROGRAM PRODUCT FOR MONITORING AT LEAST ONE ACTIVITY OF A USER.

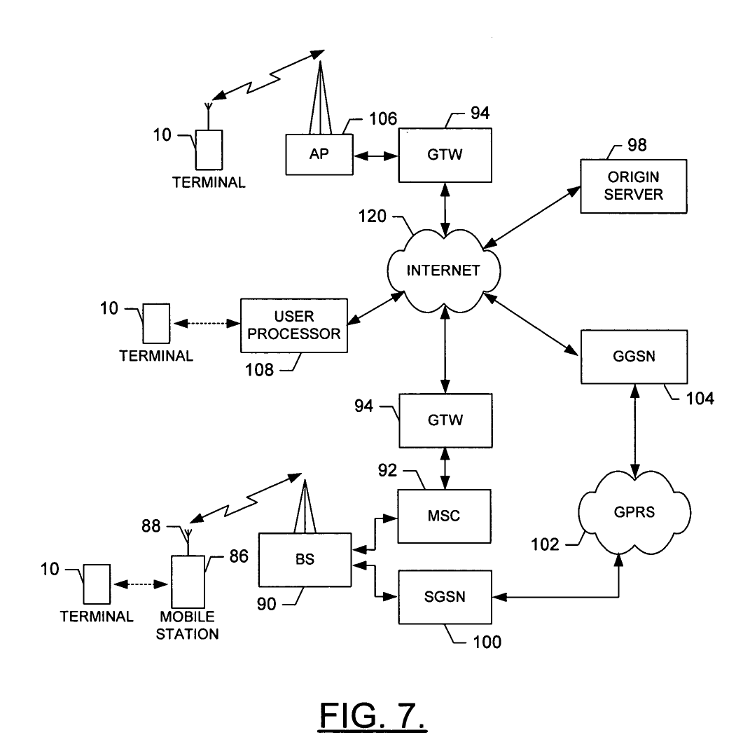

Referring to FIG. 7, an illustration of one type of system that would benefit from the terminal 10 of embodiments of the present invention is provided. The system will be primarily described in conjunction with mobile communications applications. It should be understood, however, that the system can be utilized in conjunction with a variety of other applications, both in the mobile communications industries and outside of the mobile communications industries. For example, the system of embodiments of the present invention can be utilized in conjunction with wireline and/or wireless network (e.g., Internet) applications.

As shown, the terminal 10 is capable of interfacing with a mobile station 86, such as the mobile station disclose by GB 0326387.8, in accordance with techniques such as, for example, radio frequency (RF), Bluetooth (BT), infrared (IrDA) or any of a number of different wireless networking techniques, including WLAN techniques. It should be understood, however, that although the terminal and mobile station are shown and described herein as comprising separate components of the system of FIG. 7, one or more entities may support both the terminal and the mobile station, without departing from the spirit and scope of the present invention. For example, the mobile station may include a terminal.

As shown, the mobile station 10 may include an antenna 88 for transmitting signals to and for receiving signals from a base site or base station (BS) 90. The base station is a part of one or more cellular or mobile networks that each include elements required to operate the network, such as a mobile switching center (MSC) 92. As well known to those skilled in the art, the mobile network may also be referred to as a Base Station/MSC/Interworking function (BMI). In operation, the MSC is capable of routing calls to and from the mobile station when the mobile station is making and receiving calls. The MSC can also provide a connection to landline trunks when the mobile station is involved in a call. In addition, the MSC can be capable of controlling the forwarding of messages to and from the mobile station, and can also control the forwarding of messages for the mobile station to and from a messaging center, such as short messaging service (SMS) messages to and from a SMS center (SMSC).

The MSC 92 can be coupled to a data network, such as a local area network (LAN), a metropolitan area network (MAN), and/or a wide area network (WAN). The MSC can be directly coupled to the data network. In one typical embodiment, however, the MSC is coupled to a GTW 94, and the GTW is coupled to a WAN, such as the Internet 96. In turn, devices such as processing elements (e.g., personal computers, server computers or the like) can be coupled to the mobile station 86, and thus the terminal 10, via the Internet. For example, as explained below, the processing elements can include one or more processing elements associated with an origin server 98 or the like, one of which being illustrated in FIG. 7.

The BS 90 can also be coupled to a signaling GPRS (General Packet Radio Service) support node (SGSN) 100. As is well known, the SGSN is typically capable of performing functions similar to the MSC 92 for packet switched services. The SGSN, like the MSC, can be coupled to a data network, such as the Internet 96. The SGSN can be directly coupled to the data network. In a more typical embodiment, however, the SGSN is coupled to a packet-switched core network, such as a GPRS core network 102. The packet-switched core network is then coupled to another GTW, such as a GTW GPRS support node (GGSN) 104, and the GGSN is coupled to the Internet. In addition to the GGSN, the packet-switched core network can also be coupled to a GTW 94. Also, the GGSN can be coupled to a messaging center, such as a multimedia messaging service (MMS) center. In this regard, the GGSN and the SGSN, like the MSC, can be capable of controlling the forwarding of messages, such as MMS messages. The GGSN and SGSN can also be capable of controlling the forwarding of messages for the mobile station, and thus the terminal 10, to and from the messaging center.

In addition, by coupling the SGSN 100 to the GPRS core network 102 and the GGSN 104, devices such as origin servers 98 can be coupled to the mobile station 86, and thus the terminal 10, via the Internet 96, SGSN and GGSN. In this regard, devices such as origin servers can communicate with the mobile station across the SGSN, GPRS and GGSN. For example, origin servers can provide content to the mobile station, such as in accordance with the Multimedia Broadcast Multicast Service (MBMS). For more information on the MBMS, see Third Generation Partnership Project (3GPP) technical specification 3GPP TS 22.146, entitled: Multimedia Broadcast Multicast Service (MBMS), the contents of which are hereby incorporated by reference in its entirety.

Although not every element of every possible mobile network is shown and described herein, it should be appreciated that the mobile station 86, and thus the terminal 10, can be coupled to one or more of any of a number of different networks through the BS 90. In this regard, the network(s) can be capable of supporting communication in accordance with any one or more of a number of first-generation (1G), second-generation (2G), 2.5G and/or third-generation (3G) mobile communication protocols or the like. For example, one or more of the network(s) can be capable of supporting communication in accordance with 2G wireless communication protocols IS-136 (TDMA), GSM, and IS-95 (CDMA). Also, for example, one or more of the network(s) can be capable of supporting communication in accordance with 2.5G wireless communication protocols GPRS, Enhanced Data GSM Environment (EDGE), or the like. Further, for example, one or more of the network(s) can be capable of supporting communication in accordance with 3G wireless communication protocols such as Universal Mobile Telephone System (UMTS) network employing Wideband Code Division Multiple Access (WCDMA) radio access technology. Some narrow-band AMPS (NAMPS), as well as TACS, network(s) may also benefit from embodiments of the present invention, as should dual or higher mode mobile stations (e.g., digital/analog or TDMA/CDMA/analog phones).

In addition to, or in lieu of, interfacing the terminal with a mobile station 86, the terminal 10 can be coupled to one or more wireless access points (APs) 106. The APs can comprise access points configured to communicate with the terminal in accordance with techniques such as, for example, radio frequency (RF), Bluetooth (BT), infrared (IrDA) or any of a number of different wireless networking techniques, including WLAN techniques. Additionally, or alternatively, the terminal can be coupled to one or more user processors 108. Each user processor can comprise a computing system such as personal computers, laptop computers or the like. In this regard, the user processors can be configured to communicate with the mobile station in accordance with techniques such as, for example, RF, BT, IrDA or any of a number of different wireline or wireless communication techniques, including LAN and/or WLAN techniques. One or more of the user processors can additionally, or alternatively, include a removable memory capable of storing content, which can thereafter be transferred to the terminal.

The APs 106 and the user processors 108 may be coupled to the Internet 96. Like with the MSC 92, the APs and user processors can be directly coupled to the Internet. In one advantageous embodiment, however, the APs are indirectly coupled to the Internet via a GTW 92. As will be appreciated, by directly or indirectly connecting the terminals 10 and the origin server 98, as well as any of a number of other devices, to the Internet, the terminals can communicate with one another, the origin server, etc., to thereby carry out various functions of the terminal, such as to transmit data, content or the like to, and/or receive content, data or the like from, the origin server.

According to one aspect of the present invention, all or a portion of the system of the present invention, such as all or portions of the terminal 10 generally operates under control of a computer program product (e.g., activity detection application 23). The computer program product for performing the methods of embodiments of the present invention includes a computer-readable storage medium, such as the non-volatile storage medium, and computer-readable program code portions, such as a series of computer instructions, embodied in the computer-readable storage medium.

In this regard, FIG. 5 is a flowchart of methods, systems and program products according to the invention. It will be understood that each block or step of the flowchart, and combinations of blocks in the flowchart, can be implemented by computer program instructions. These computer program instructions may be loaded onto a computer or other programmable apparatus to produce a machine, such that the instructions which execute on the computer or other programmable apparatus create means for implementing the functions specified in the flowchart block(s) or step(s). These computer program instructions may also be stored in a computer-readable memory that can direct a computer or other programmable apparatus to function in a particular manner, such that the instructions stored in the computer-readable memory produce an article of manufacture including instruction means which implement the function specified in the flowchart block(s) or step(s). The computer program instructions may also be loaded onto a computer or other programmable apparatus to cause a series of operational steps to be performed on the computer or other programmable apparatus to produce a computer implemented process such that the instructions which execute on the computer or other programmable apparatus provide steps for implementing the functions specified in the flowchart block(s) or step(s).

Accordingly, blocks or steps of the flowchart supports combinations of means for performing the specified functions, combinations of steps for performing the specified functions and program instruction means for performing the specified functions. It will also be understood that each block or step of the flowchart, and combinations of block(s) or step(s) in the flowchart, can be implemented by special purpose hardware-based computer systems which perform the specified functions or steps, or combinations of special purpose hardware and computer instructions.

Many modifications and other embodiments of the invention will come to mind to one skilled in the art to which this invention pertains having the benefit of the teachings presented in the foregoing descriptions and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

1. A terminal comprising:
data transfer circuitry capable of at least one of transferring data and receiving data;
tactile circuitry capable of measuring at least one condition of a user; and
at least one conductor alternately coupled to both the data transfer circuitry and the tactile circuitry, the at least one conductor being capable of communicating with the data transfer circuitry to function as an antenna, and capable of alternately communicating with the tactile circuitry to function as a tactile interface.

2. A terminal according to claim 1 further comprising:
switching logic coupled to the tactile circuitry, data transfer circuitry and at least one conductor, wherein the switching logic is capable of operating to permit the data transfer circuitry to communicate with the at least one conductor functioning as an antenna, or permit the tactile circuitry to communicate with the at least one conductor functioning as a tactile interface.

3. A terminal according to claim 1 further comprising:
a controller coupled to the switching logic and capable of controlling operation of the switching logic.

4. A terminal according to claim 3, wherein the controller is capable of controlling the switching logic to permit the tactile circuitry to communicate with the at least one conductor functioning as a tactile interface when the tactile circuitry is set to operate.

5. A terminal according to claim 4, wherein the controller is capable of determining when the tactile circuitry is set to operate based upon at least one of user input and a context of at least one of the terminal and user.

6. A terminal according to claim 5, wherein the controller is further capable of determining the context of at least one of the terminal and user based upon at least one of data representative of a change in a radio frequency field proximate the conductors, and data representative of a change in capacitance across at least one pair of conductors.

7. A terminal according to claim 1 further comprising:
a housing for supporting the at least one conductor.

8. A terminal according to claim 1, wherein the tactile circuitry is capable of communicating monitoring signals to a portion of the user's body via the at least one conductor operating as a tactile interface such that at least one condition of the user is capable of being computed in response to communication of the monitoring signals.

9. A terminal according to claim 8, wherein the tactile circuitry is capable of communicating monitoring signals to a portion of the user's body such that a resistance to the communication of the monitoring signals is capable of being computed, and such that at least one parameter associated with the user's body fat is capable of being computed based upon the resistance.

10. A terminal according to claim 9, wherein the at least one parameter comprises a percent body fat.

11. A terminal according to claim 9, wherein the at least one parameter comprises at least one of a fat mass and a total body water.

12. A terminal according to claim 8, wherein the tactile circuitry is capable of communicating monitoring signals comprising one of sinusoidal current and voltage signals.

13. A terminal according to claim 8, wherein the tactile circuitry is capable of communicating monitoring signals comprising one of square wave current and voltage signals.

14. A terminal according to claim 8, wherein the tactile circuitry is capable of communicating monitoring signals comprising one of a current and voltage signal having a frequency of at least 50 kHz.

15. A terminal according to claim 8, wherein the tactile circuitry is capable of communicating monitoring signals comprising a current signal having an amperage less than 1 mA.

16. A terminal according to claim 8, wherein the tactile circuitry is capable of maintaining the monitoring signals at one of a substantially constant amperage and voltage.

17. A method of monitoring at least one condition of a user, the method comprising:
providing a terminal comprising data transfer circuitry, tactile circuitry, and at least one conductor alternately coupled to the data transfer circuitry and the tactile circuitry, the at least one conductor being capable of alternately functioning as an antenna or a tactile interface; and
switching operation of the at least one conductor to function as a tactile interface to thereby permit the tactile circuitry to communicate with the at least one conductor functioning as a tactile interface to measure at least one condition of the user.

18. A method according to claim 17, wherein switching operation comprises switching operation of the at least one conductor to function as a tactile interface when the tactile circuitry is set to operate.

19. A method according to claim 18 further comprising:
determining when the tactile circuitry is set to operate based upon at least one of user input and a context of at least one of the terminal and user.

20. A method according to claim 19, wherein determining when the tactile circuitry is set to operate based upon a context comprises determining the context of at least one of the terminal and user based upon at least one of data representative of a change in a radio frequency field proximate the conductors, and data representative of a change in capacitance across at least one pair of conductors.

21. A method according to claim 17, wherein providing a terminal comprises providing a terminal further comprising a housing for supporting the at least one conductor.

22. A method according to claim 17 further comprising:
communicating monitoring signals from the tactile circuitry to a portion of the user's body via the at least one conductor operating as a tactile interface; and
computing at least one condition of the user in response to communication of the monitoring signals.

23. A method according to claim 22 further comprising:
computing a resistance to the communication of the monitoring signals,
wherein computing at least one condition comprises computing at least one parameter associated with the user's body fat based upon the resistance.

24. A method according to claim 23, wherein computing at least one parameter comprises computing a percent body fat.

25. A method according to claim 23, wherein computing at least one parameter comprises computing at least one of a fat mass and a total body water.

26. A method according to claim 22, wherein communicating monitoring signals comprises communicating monitoring signals comprising one of sinusoidal current and voltage signals.

27. A method according to claim 22, wherein communicating monitoring signals comprises communicating monitoring signals comprising one of square wave current and voltage signals.

28. A method according to claim 22, wherein communicating monitoring signals comprises communicating monitoring signals comprising one of a current and voltage signal having a frequency of at least 50 kHz.

29. A method according to claim 22, wherein communicating monitoring signals comprises communicating monitoring signals comprising a current signal having an amperage less than 1 mA.

30. A method according to claim 22, wherein communicating monitoring signals further comprises maintaining the monitoring signals at one of a substantially constant amperage and voltage.

31. A computer program product for monitoring at least one condition of a user, the computer program product adapted to operate within a terminal comprising data transfer circuitry, tactile circuitry, and at least one conductor alternately coupled to the data transfer circuitry and the tactile circuitry, the at least one conductor being capable of alternately functioning as an antenna or a tactile interface, wherein the computer program product comprises at least one computer-readable storage medium having computer-readable program code portions stored therein, the computer-readable program code portions comprising:
a first executable portion for switching operation of the at least one conductor to function as a tactile interface to thereby permit the tactile circuitry to communicate with the at least one conductor functioning as a tactile interface to measure at least one condition of the user.

32. A computer program product according to claim 31, wherein the first executable portion is adapted to switch operation of the at least one conductor to function as a tactile interface when the tactile circuitry is set to operate.

33. A computer program product according to claim 32 further comprising:
a second executable portion for determining when the tactile circuitry is set to operate based upon at least one of user input and a context of at least one of the terminal and user.

34. A computer program product according to claim 33, wherein the second executable portion is adapted to determine the context of at least one of the terminal and user based upon at least one of data representative of a change in a radio frequency field proximate the conductors, and data representative of a change in capacitance across at least one pair of conductors.

35. A computer program product according to claim 31 further comprising:
a second executable portion for controlling the tactile circuitry to communicate monitoring signals to a portion of the user's body via the at least one conductor operating as a tactile interface; and
a third executable portion for computing at least one condition of the user in response to communication of the monitoring signals.

36. A computer program product according to claim 35 further comprising:
a fourth executable portion for computing a resistance to the communication of the monitoring signals,
wherein the third executable portion is adapted to compute at least one parameter associated with the user's body fat based upon the resistance.

37. A computer program product according to claim 36; wherein the third executable portion is adapted to compute a percent body fat.

38. A computer program product according to claim 36, wherein the third executable portion is adapted to compute at least one of a fat mass and a total body water.

39. A computer program product according to claim 35, wherein the second executable portion is adapted to control the tactile circuitry to communicate monitoring signals comprising one of sinusoidal current and voltage signals.

40. A computer program product according to claim 35, wherein the second executable portion is adapted to control the tactile circuitry to communicate monitoring signals comprising one of square wave current and voltage signals.

41. A computer program product according to claim 35, wherein the second executable portion is adapted to control the tactile circuitry to communicate monitoring signals comprising one of a current and voltage signal having a frequency of at least 50 kHz.

42. A computer program product according to claim 35, wherein the second executable portion is adapted to control the tactile circuitry to communicate monitoring signals comprising a current signal having an amperage less than 1 mA.

43. A computer program product according to claim 35, wherein the second executable portion is adapted to further control the tactile circuitry to maintain the monitoring signals at one of a substantially constant amperage and voltage.
